(19) United States
(12) Patent Application Publication
(10) Pub. No.: US 2009/0088723 A1
(43) Pub. Date: Apr. 2, 2009

(54) APPARATUS AND METHODS FOR TREATING PSEUDOANEURYSMS

(75) Inventors: Farhad Khosravi, Los Altos Hills, CA (US); Syed Hasan Askari, San Jose, CA (US)

Correspondence Address: Vista IP Law Group LLP, 2040 MAIN STREET, 9TH FLOOR, IRVINE, CA, 92614, US

(73) Assignee: AccessClosure, Inc.

(21) Appl. No.: 12/167,985

(22) Filed: Jul. 3, 2008

Related U.S. Application Data
(60) Provisional application No. 60/976,351, filed on Sep. 28, 2007.

(51) Int. Cl. A61M 31/00

(52) U.S. Cl. 604/508; 604/60

(57) ABSTRACT

Apparatus and methods are provided for treating pseudoaneurysms using freeze-dried hydrogel particles that expand and/or absorb fluid within a pseudoaneurysm. An apparatus includes an elongate tubular member including a proximal end, a distal end sized for introduction through tissue into a pseudoaneurysm or other body cavity, and a lumen communicating with an outlet on the distal end. The particles are provided within the lumen and dischargeable through the outlet, e.g., using a plunger or other actuator. The apparatus may include one or more of a bled-back channel, flow sensor, pressure sensor, and markers for monitoring the distal end during introduction into the pseudoaneurysm.

The present application claims benefit of co-pending provisional application Ser. No. 60/976,351, filed Sep. 28, 2007, the entire disclosure of which is expressly incorporated by reference herein.

### FIELD OF THE INVENTION

The present invention relates generally to apparatus and methods for delivering materials into a patient's body, and, more particularly, to apparatus and methods for delivering polymeric particles and/or other materials into body lumens or cavities, e.g., for treating pseudoaneurysms.

### BACKGROUND

A pseudoaneurysm, also known as a “false aneurysm,” results from disruption or injury of a vessel wall, creating a pulsatile build-up of blood and blood clot in communication with the lumen of the vessel. The bleeding to the pseudoaneurysm from the vessel may be contained, at least temporarily, by a blood clot or surrounding tissue structures.

Pseudoaneurysms often result from an accident or a blood vessel being damaged during a surgical procedure, although disease may also contribute to pseudoaneurysm formation. Pseudoaneurysms may heal naturally by thrombosis and need no treatment. However, there is a risk that the pseudoaneurysm may rupture and bleed into the body, such that it is desirable to treat the pseudoaneurysm before such an event occurs. While a pseudoaneurysm may be treated with surgery, it may be useful to treat a pseudoaneurysm with less invasive techniques, e.g., that may be less traumatic for the patient.

### SUMMARY OF THE INVENTION

The present invention is directed to apparatus and methods for delivering materials into a patient's body. More particularly, the present invention is directed to apparatus and methods for delivering polymeric particles and/or other materials into body lumens or cavities, e.g., for treating a pseudoaneurysm.

In accordance with one embodiment, an apparatus is provided that includes a delivery lumen for carrying an expandable material therein that is dischargeable from the apparatus through an outlet communicating with the delivery lumen. The apparatus may include a plunger and/or other actuator for discharging the material from the outlet of the apparatus. The apparatus may also include one or more elements for positioning the apparatus and/or for imaging the apparatus during introduction into a patient's body. In one embodiment, the apparatus may include a bleed-back channel for receiving fluids within the patient contacted by the device. In particular, blood received in the bleed-back channel may indicate that the device is located at a pseudoaneurysm or other body lumen or cavity intended for treatment. In alternative embodiments, the apparatus may include one or more echogenic and/or radiopaque markers for monitoring the apparatus using ultrasound and/or x-ray imaging. In another embodiment, the apparatus may include a flow sensor that detects laminar and/or turbulent flow of fluids adjacent the apparatus. In particular, a sensor may detect a turbulent flow condition that may indicate when the apparatus is located adjacent or within a pseudoaneurysm. In addition or alternatively, the sensor may detect laminar flow when blood is no longer flowing to and/or within the pseudoaneurysm.

The material carried by the apparatus may include one or more polymeric components, e.g., a bolus of expandable particles. In one embodiment, the material includes xerogel, e.g., freeze-dried hydrogel. The xerogel may rapidly swell when exposed to an aqueous environment, such as within a pseudoaneurysm, and may swell to multiple times its initial mass. The xerogel may also expand to multiple times its initial volume.

In accordance with another embodiment, a method is provided for treating a pseudoaneurysm in communication with a vessel. A delivery device may be inserted through tissue and advanced toward a pseudoaneurysm or other body lumen or cavity being treated. Optionally, the device may include one or more elements, e.g., to locate the device relative to the pseudoaneurysm or other body lumen or cavity. For example, the device may include a bleed-back channel, and blood exiting from the bleed-back channel may indicate that the device is located in the pseudoaneurysm, i.e., when the bleed-back channel is in communication with the pseudoaneurysm. Alternatively, a change in pressure or flow condition may be detected to indicate when the device is disposed adjacent to or within the pseudoaneurysm.

Once the device is inserted into the pseudoaneurysm, an expandable material, e.g., a plurality of particles, may be delivered from the device into the pseudoaneurysm. In one embodiment, before delivering the absorption agent, pressure may be applied to the patient's skin upstream to the vessel communicating with the pseudoaneurysm to reduce or substantially discontinue flow through the vessel adjacent the pseudoaneurysm. This may reduce the risk of the expandable material exiting from the pseudoaneurysm into the vessel. After the material is delivered into the pseudoaneurysm, the material may absorb blood and/or other fluid within the pseudoaneurysm and expand, e.g., to substantially block flow of fluid between the vessel and the pseudoaneurysm, to substantially fill the pseudoaneurysm, and/or to contain blood, clot, and/or other material within the pseudoaneurysm. In another embodiment, the initial size of the particles may be larger than the aneurysm opening such that the particles pose essentially no risk of outflow from the pseudoaneurysm into the vessel.

Optionally, the particles may be coated with and/or otherwise include varying amounts of materials, such as thrombogin or other pro-thrombotic materials. Thus, the blood within the pseudoaneurysm may clot and seal the aneurysm substantially immediately on contact with the particles and thereby may not allow blood flow out from the pseudoaneurysm into the vessel. In addition or alternatively, the polymer may be radiopaque and/or echogenic.

In another embodiment, the polymer may be injected in the form of a flowable material, e.g., a putty-consistent material and, when pressure is applied, a superabsorbent elongate bead, rod, wire, or other extrusion of the flowable material may be extruded from the delivery device or otherwise injected at the site of the pseudoaneurysm.

The material may slowly degrade within the body over a period of time, ranging from a day to one or more months, or the agent may be substantially non-degradable such that the material may not degrade within about one to two years.

In accordance with still another embodiment, an apparatus for treating a pseudoaneurysm or other body cavity is provided that includes an elongate body including a proximal end, a distal end sized for introduction through tissue into a pseudoaneurysm or other body cavity, and a lumen communicating with an outlet on the distal end. A plurality of particles may be provided within the lumen and dischargeable through the outlet into a pseudoaneurysm or other body cavity. The particles, e.g., formed from xerogel, such as freeze-dried hydrogel, may be configured for absorbing fluid within the pseudoaneurysm or other body cavity to cause the particles to expand to substantially fill the pseudoaneurysm or other body cavity.

In accordance with yet another embodiment, an apparatus is provided for treating a pseudoaneurysm or other body cavity that includes an elongate body including a proximal end, a distal end sized for introduction into a pseudoaneurysm or other body cavity, a delivery lumen extending between the proximal and distal ends, and a bleed-back channel extending between the distal end a proximal opening. A plurality of particles may be provided within the delivery lumen and dischargeable through an outlet at the distal end, e.g., a xerogel, such as a freeze-dried hydrogel, that absorbs fluid within the pseudoaneurysm or other body cavity to cause the particles to expand to substantially fill the pseudoaneurysm or other body cavity. The apparatus may also include an actuator operable from the proximal end for discharging the particles from the outlet into a pseudoaneurysm or other body cavity, e.g., a plunger depressible to discharge the particles from the outlet.

In accordance with still another embodiment, a method is provided for treating a pseudoaneurysm or other body lumen or cavity within a patient's body that includes inserting a distal end of a delivery device into tissue having the cavity therein; inserting the distal end of the delivery device into the cavity; delivering from the delivery device into the cavity, the particles absorbing fluid and expanding within the pseudoaneurysm.

In accordance with yet another embodiment, a method is provided for treating a pseudoaneurysm communicating with a vessel that includes introducing a delivery device into tissue adjacent the pseudoaneurysm; monitoring introduction of the distal end using an element on the distal end until the element provides an indication that the distal end is within the pseudoaneurysm; and delivering the particles from the delivery device into the pseudoaneurysm, the particles absorbing fluid and/or expanding within the pseudoaneurysm. The particles may include xerogel, e.g., freeze-dried hydrogel, that may expand upon absorbing fluid within the pseudoaneurysm to substantially fill the aneurysm, substantially isolate the pseudoaneurysm from the vessel, relieve pressure within the pseudoaneurysm, contain blood, clot, other materials within the pseudoaneurysm, and/or deliver diagnostic and/or therapeutic agents into the pseudoaneurysm.

Other aspects and features of the present invention will become apparent from consideration of the following description taken in conjunction with the accompanying drawings.

### BRIEF DESCRIPTION OF THE DRAWINGS

The drawings illustrate exemplary embodiments in which:

FIG. 1 is a cross-sectional view of an exemplary embodiment of an apparatus for delivering expandable particles into a patient's body.

FIGS. 2A-2D are partial cross-sectional views of a patient's body, showing a method for treating a pseudoaneurysm using in the apparatus of FIG. 1.

FIGS. 3A-3C are side views of another exemplary embodiment of an apparatus for delivering expandable particles into a patient's body.

### DETAILED DESCRIPTION OF THE EXEMPLARY EMBODIMENTS

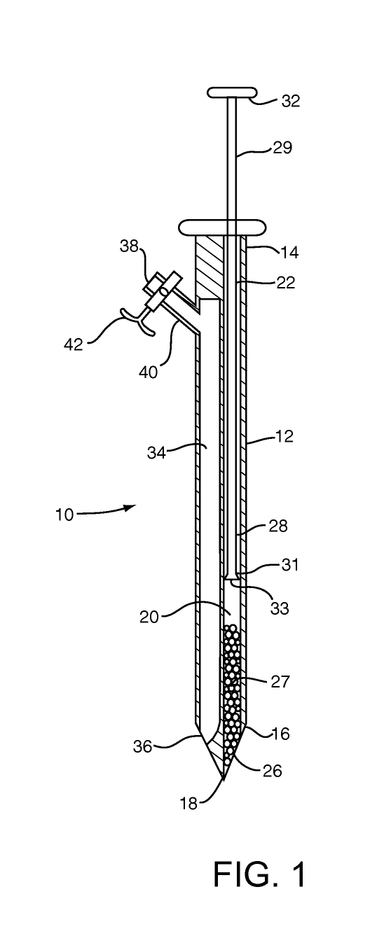

Turning to the drawings, FIG. 1 shows an exemplary embodiment of an apparatus 10 for delivering expandable particles and/or other material into a patient's body, e.g., to expand and/or absorb fluid within a pseudoaneurysm or other body lumen or cavity. Generally, the apparatus 10 includes an elongate tubular member 12, particles and/or other material 27 carried by the tubular member 12, and a plunger or other actuator 28 for delivering the material 27 from the tubular member 12.

The tubular member 12 generally includes a proximal end 14, a distal end 16, and one or more lumens 20, 34 extending between the proximal and distal ends 14, 16. The tubular member 12 may be sized and/or shaped for percutaneous insertion into tissue, e.g., having a length between about five and thirty centimeters (5-30 cm) or between about ten and twenty five centimeters (10-25 cm), and an outer diameter between about 0.7 and five millimeters (0.7-5 mm), or between about one and four millimeters (1-4 mm) As shown, the distal end 16 includes a sharpened distal tip 18, e.g., for puncturing skin and/or facilitating advancement of the device 10 through tissue.

The tubular member 12 may be formed from a substantially rigid body, e.g., having sufficient column strength such that the tubular member 12 may be advanced through tissue, e.g., without additional supporting devices. Alternatively, the tubular member 12 may be semi-rigid or substantially flexible, e.g., to permit different orientations for facilitating positioning of the apparatus 10. In such alternatives, the apparatus 10 may include one or more other instruments (not shown), e.g., an internal obturator or an external sheath or introducer (not shown), which may facilitate advancement of the apparatus 10 through tissue. Exemplary materials for the tubular member 12 include metal, such as stainless steel, plastic, or composite materials.

As shown in FIG. 1, the apparatus 10 includes a pair of lumens 20, 34 extending between the proximal and distal ends 14, 16. A first or delivery lumen 20 may extend longitudinally from the proximal end 14 to the distal end 16 of the elongate body 12, e.g., for carrying the material 27 and/or plunger 28, as described further elsewhere herein. The delivery lumen 20 may include a first port or inlet 22 at the proximal end 14 of the tubular member 12 and a second port or outlet 26 at or near the distal end 16 of the tubular member 12. Alternatively, the delivery lumen 20 may extend only partially from the distal end 16 towards the proximal end 14, e.g., to provide a chamber of sufficient size to receive a desired bolus of material 27. As shown, the material 27 is carried within the delivery lumen 20 immediately adjacent the outlet 26, although the material 27 may be disposed more proximally within the delivery lumen 20, if desired.

The plunger 28 extends longitudinally through the inlet 22 and into the delivery lumen 20, and generally includes a proximal end 29 protruding from the inlet 22 and a distal end 31 disposed within the delivery lumen 20, e.g., initially adjacent or otherwise proximal to the material 27. The plunger 28 is slidable within the delivery lumen 20 and may be operated to advance and/or retract therein, e.g., using a handle 32 on the proximal end 29 of the plunger 28. The plunger 28 also includes a distal end 31, e.g., including a piston or other enlarged region 33 that extends across the delivery lumen 20 and/or slidably engages the wall of the delivery lumen 20. Thus, when the plunger 28 is depressed, the enlarged region 33 causes material 27 in the delivery lumen 20 to be discharged from the outlet 26. Alternatively, other actuators may be provided instead of or in addition to the plunger 28, e.g., for manually or automatically advancing a piston or enlarged region, e.g., similar to enlarged region 33 to discharge material 27 from the delivery lumen 20.

In addition, as shown in FIG. 1, the tubular member 12 includes a bleed-back lumen or channel 34 that extends longitudinally, e.g., between a distal opening or port 36 and a proximal opening or port 38. In one embodiment, the tubular member 12 may include a side port 40 on the proximal end 14 having the proximal opening 38 therein. Optionally, the side port 40 may include a shut-off valve 42, e.g., which may manually opened and/or closed, for controlling flow of fluid within the bleed-back channel 34 and/or through the proximal opening 38 by adjusting the shut-off valve 42 between an open position and a closed position. In an alternative embodiment, the proximal opening 38 may be positioned on a surface along the length of the tubular member 12, or the proximal opening 38 may be positioned at the proximal end 14 of the tubular member 12 (not shown). The bleed-back channel 34 may be positioned adjacent the delivery lumen 20 in the tubular member 12, as shown in FIG. 1. Alternatively, the bleed-back channel 34 and the delivery lumen 20 may be arranged coaxially, such that one circumferentially surrounds the other.

Optionally, the apparatus 10 may include one or more additional features, e.g., in addition to or as an alternative to the bleed-back channel 34. For example, in one embodiment, one or more echogenic elements (not shown) may be provided on the end portion 16, e.g., to facilitate imaging the apparatus 10 using external ultrasound imaging equipment. The echogenic elements may include, for example, bubbles, particles, or discontinuities on a surface of the distal end 16. In an alternative embodiment, one or more radiopaque markers, such as one or more circumferential bands (not shown), may be provided on the distal end 16, e.g., to facilitate imaging the apparatus 10 using fluoroscopy or other x-ray imaging equipment. Such markers may be embedded in or printed on a surface of the tubular member 12, crimped around the tubular member 12, and the like. In yet another embodiment, a series of hashes or other marks (not shown) may be vertically aligned along a length of the tubular member 12. For example, a series of marks scaled from the distal tip 18 may be provided that correspond to the distance to the distal tip 18 from each mark. Such marks may be used to indicate a distance that the distal tip 18 of the apparatus 10 has been inserted into a patient based on the marks exposed above the patient's skin.

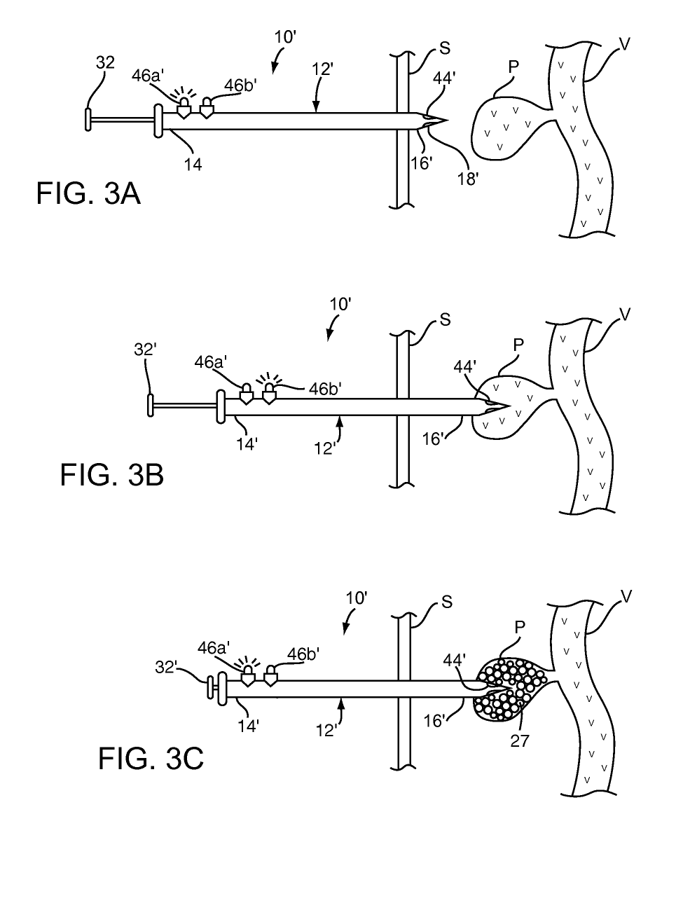

Optionally, as shown in FIGS. 3A-3C, instead of or in addition to the bleed-back channel 34, an apparatus 10′ may include one or more sensors 44′ for facilitating monitoring the apparatus 10′ during insertion into a patient's body (not shown). For example, the apparatus 10′ may include a tubular member 12′ similar to other embodiments herein, but including a flow sensor, a Doppler sensor, or a pressure sensor (not shown), on the distal end 16. In one embodiment, the sensor 44′ may detect and/or indicate when the distal end 16′ is disposed adjacent to or within a pseudoaneurysm P based upon fluid flowing therein.

For example, blood flowing through a vessel in communication with a pseudoaneurysm may undergo turbulent flow as it enters, exits, and/or flows within the pseudoaneurysm. In contrast, blood in an uninterrupted vessel may exhibit substantially laminar flow. Thus, a turbulent flow condition may indicate that that the distal end 16′ is located near or within the pseudoaneurysm as opposed to the adjacent tissue or vessel. Also, when blood ceases flowing into, from, and/or within the pseudoaneurysm, the blood in the adjacent vessel may resume laminar flow. Thus, a laminar flow condition or a no flow condition may indicate that blood is no longer entering the pseudoaneurysm from the vessel, as described further elsewhere herein.

Similarly, if the sensor 44′ is a pressure sensor, a relatively low pressure may indicate that the distal end 16′ is within tissue, while an increase in pressure may indicate that the distal end 16′ is within the pseudoaneurysm P.

The sensor 44′ may be coupled to an output device 44,′ e.g., one or more lights or other indicators, a display, and the like, e.g., by one or more leads (not shown), for providing an output based upon the conditions detected by the sensor 44.′ For example, as shown in FIGS. 3A-3C, one or more LEDs or other lights 46′ may be provided on the proximal end 14′ of the tubular member 12.′ Alternatively, the sensor 44′ may be coupled to instrumentation separate from the apparatus 10,′ e.g., to a display (not shown), which may generate images based upon information from the sensor 44.′

For example, when the sensor 44′ detects laminar flow (or no flow), a first light 46a′ may be lit, as shown in FIG. 3A. As the distal end 16′ of the tubular member 12′ approaches or enters a pseudoaneurysm P, the sensor 44′ may detect turbulent flow, and activate a second light 46b′ (and deactivate the first light 46a′), as shown in FIG.3B. If flow in the pseudoaneurysm P discontinues, e.g., due filling with material 27, as described elsewhere herein, the second light 46b′ may be deactivated and the first light 46a′ activated when the sensor 44′ no longer detects turbulent flow, as shown in FIG. 3C. It will be appreciated that more simple or complicated display arrangements may be provided instead of the lights 46a′ depending upon the information desired or available from the sensor 44.′

Returning to FIG. 1 (although applicable to other embodiments herein), the material 27 may include a bolus of particles capable of expanding and/or absorbing fluid, e.g., once exposed within an aqueous environment. Generally, the material 27 may be biocompatible, fast-swelling, and/or biodegradable, and, optionally, may also include pro-thrombotic material. Exemplary materials include sugar, starch, lactic acids, glycolic acids, acrylates, and polymeric materials.

In one embodiment, the material 27 may be a PEG polymeric material, e.g., a xerogel or hydrogel formed exclusively by the reaction of high molecular weight PEG-esters with PEG-amines, such as a freeze-dried hydrogel or other xerogel, having a density between about 0.05 and 0.90 grams per cubic centimeter (g/cc). As used herein, “xerogel” refers to a hydrogel material in a dehydrated state, which may be achieved by freeze-drying the hydrogel or by other methods. The term “hydrogel” may be used generically or may refer to the material in a hydrated state. Density, along with the precursor components and/or other process parameters, may affect one or more properties of the xerogel material, e.g., rate of swelling, magnitude of swelling, compressive modulus, and the like. For example, the xerogel may rapidly swell when exposed to an aqueous environment, such as when delivered within a pseudoaneurysm, e.g., swelling between about two hundred and three thousand percent (200-3000%) of the initial mass within about five to sixty (5-60) seconds (“rate of swelling”). In addition or alternatively, the xerogel may expand between about two and fifty (2-50) times in volume from its dehydrated state after being formed to its fully hydrated state (“magnitude of swelling”). Once hydrated, the hydrogel may be absorbed or otherwise degrade within the body over a period of time, e.g., between about one and ninety (1-90) days or between about five and sixty (5-60) days. Alternatively, the hydrogel may be substantially non-degradable, i.e., may not substantially degrade within about one to two years in a physiological environment. Additional information on materials that may be used and/or methods for making and/or using them are disclosed in U.S. Pat. Nos. 6,152,943, 6,165,201, 6,179,862, 6,514,534, and 6,379,373, and in co-pending applications Ser. No. 09/776,120 filed Feb. 2, 2001, Ser. No. 10/010,715 filed Nov. 9, 2001, Ser. No. 10/068,807 filed Feb. 5, 2002, Ser. No. 10/454,362, filed Jun. 4, 2003, and Ser. No. 11/465,791, filed Aug. 18, 2006. The disclosures of these references and any others cited therein are expressly incorporated by reference herein.

The material 27 may be initially prepared in sheet form, e.g., using the methods disclosed in application Ser. No. 11/465,791, incorporated by reference herein. A plurality of particles may then be created from the resulting sheet, for example, by successively punching individual particles or simultaneously punching multiple particles out of the sheet, for example, using a hole punch having desired dimensions for the resulting particles, e.g., one or more diameters between about 0.5-10 millimeters. The diameters of the particles may be substantially uniform or may vary, if desired. Alternatively, the particles may be cut from the sheet using other methods, such as die-cutting, laser cutting, and the like. Optionally, the particles may be synthesized using conventional particle manufacturing technologies, such as oil/water mixture, which may include adjusting the mixture ratio and stirring rate in such a way that desired particle sizes are obtained. The concentration of the solvent and the particles, along with the stirring speed, may be adjusted to obtain a desired final particle size. The particles may then be filtered and dried to obtain xerogel particles.

The porosity of the particles may be adjusted by freeze-drying, or any other process known in the art. Adjusting the porosity of the particles may also adjust the rate at which the particles expand and/or absorb fluid. More specifically, the porosity of the particles may be adjusted so the rate at which the particles absorb bodily fluids is extremely rapid, e.g., having a time to substantial completion of absorption of less than about one and ninety seconds. In another embodiment, the particles may be treated with saline, e.g., to produce a hydrogel, after which the hydrogel may then be dried back to a xerogel state.

Alternatively, the material 27 may be provided as a flowable material within the delivery lumen 20. For example, the material 27 may be a paste or putty-consistent material, e.g., that includes xerogel particles disposed within an inert carrier material. Thus, instead of a bolus of separate particles, an elongate bead, rod, or other extrusion of the material 27 may extruded or otherwise injected from the delivery device 10

The material 27 may be loaded into the delivery lumen 20 of the tubular member 12 during manufacturing or otherwise before the apparatus 10 is delivered to a user. Alternatively, the material 27 may be provided separately from the apparatus 10, e.g., within a bottle or other container, such that a desired amount may be loaded into the delivery lumen 20 immediately before use. Thus, the user may select the size of the bolus desired, e.g., based upon the specific anatomy encountered, and load the desired bolus, e.g., by pouring into the outlet 26 or a side port (not shown) communicating with the delivery lumen 20, inserting the distal end 16 into a container to force material 27 into the outlet 26, or otherwise loading the material 27 into the delivery lumen 20. Alternatively, multiple apparatus 10 (not shown) may be provided to a user, each having different sizes of boluses such that the user may select the appropriate size bolus corresponding to the actual anatomy encountered.

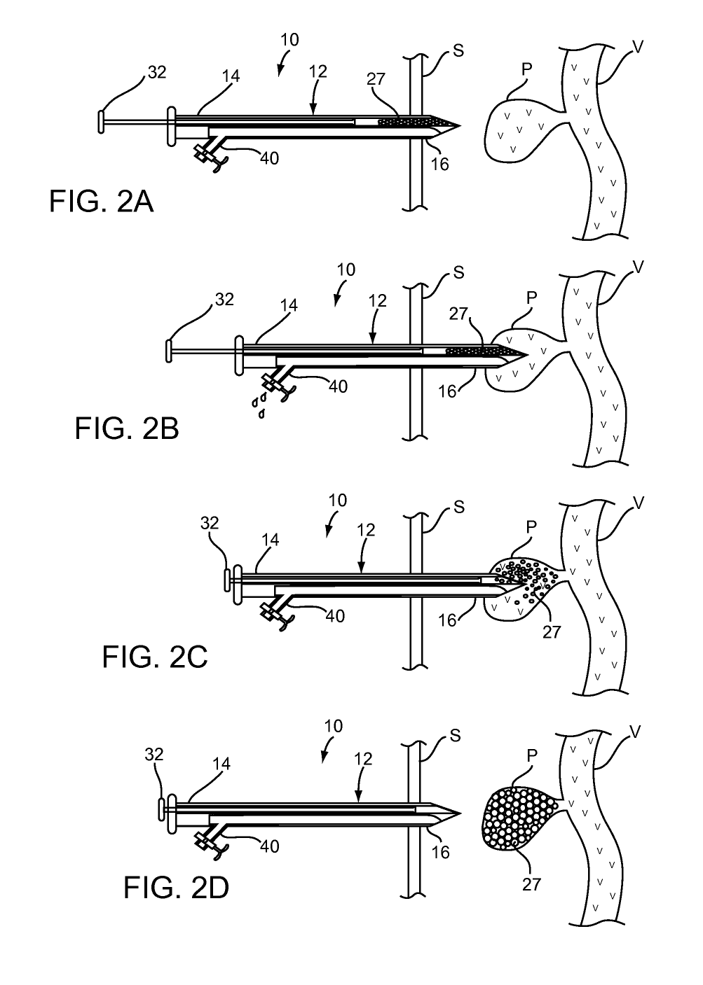

During use, as shown in FIGS. 2A-2D, the apparatus 10 may be used to treat a lumen or cavity within a patient's body, e.g., a pseudoaneurysm P originating from an injured vessel V. Referring first to FIG. 2A, the pseudoaneurysm P may be identified and/or located within a patient, e.g., using ultrasound, x-ray, or other imaging methods. The distal tip 18 of the apparatus 10 may be inserted through the patient's skin S and any intervening tissue towards the pseudoaneurysm P, as shown in FIG. 2A. For example, with the distal tip 18 sharpened, the distal tip 18 may be percutaneously directed through the skin S and advanced through the intervening tissue. Alternatively, the apparatus 10, e.g., with a blunt distal tip (not shown) may be introduced through a catheter, sheath, cannula, and the like that has already been placed between the skin S and the pseudoaneurysm P. The apparatus 10 may be manipulated from the proximal end 14 as the distal tip 18 is advanced, i.e., having sufficient column strength to prevent buckling or undesired deflection of the distal tip 18. Alternatively, if the tubular member 12 is semi-rigid or flexible, the apparatus 10 may include an obturator or other support member (not shown) coupled thereto, e.g., inserted into a lumen (also not shown) extending between the proximal and distal ends 14, 16, which may support the apparatus 10 during introduction. Optionally, the support member may be removed before delivering the material 27.

Turning to FIG. 2B, as the apparatus 10 is advanced into the patient, the distal end 16 may be located and/or imaged inside the patient, e.g., to confirm that the distal end 16 is directed towards and inserted into the pseudoaneurysm P. The distal tip 18 may penetrate through the wall of the pseudoaneurysm P such that the outlet 26 and distal opening 36 are disposed within the pseudoaneurysm P. The distal tip 18 may be positioned away from the mouth of the pseudoaneurysm P, e.g., to reduce the risk of particles escaping from the pseudoaneurysm into the vessel V.

In one embodiment, the bleed-back channel 34 may be used to locate the end portion 16 within the pseudoaneurysm P. As shown in FIG. 2A, the valve 42 may be placed in the open position such that fluid entering the bleed-back channel 34 from the distal opening 36 is free to pass through the bleed-back channel 34 and exit the proximal opening 38. Thus, as shown in FIG. 2B, when the distal end 16 of the tubular member 12 enters the pseudoaneurysm P, blood from the pseudoaneurysm P may enter the distal opening 36 of the bleed-back channel 34 and flow through the bleed-back channel 34 to the proximal opening 38 and exit the side port 40. A user observing the blood exiting from the proximal opening 38 is then alerted that the distal end 16 is located in the pseudoaneurysm P site. The valve 42 may then be moved to the closed position to prevent additional blood or other material from flowing through the bleed-back channel 34, as shown in FIG. 2C.

In addition or alternatively, the distal end 16 may be monitored using other methods. For example, ultrasound imaging may be used to identify one or more echogenic elements (not shown) on the distal end 16 to facilitate inserting the distal end 16 towards and into the pseudoaneurysm P. Alternatively, fluoroscopic or other x-ray imaging may be used to locate the pseudoaneurysm P and/or the apparatus 10, e.g., to identify one or more radiopaque markers (not shown) on the distal end 16. Radiopaque contrast may be injected upstream of the vessel V to facilitate determining the relative location of the vessel V, the pseudoaneurysm P, and the apparatus 10.

In another alternative, shown in FIGS. 3A-3C, the apparatus 10′ may include one or more sensors 44,′ which may detect changes in flow and/or pressure, e.g., to indicate that the distal portion 16 has entered a region of turbulent flow and/or increased pressure, which may correspond to the pseudoaneurysm P.

After the distal end 16 of the apparatus 10 is inserted into the pseudoaneurysm P, the plunger 28 may be depressed to advance the material 27 from the delivery lumen 20 out the outlet 26, as shown in FIG. 2C. For example, the material 27 may be a bolus including a plurality of separate particles that may be delivered from the apparatus 10, or the material 27 may be a paste or other flowable material that may be extruded from the apparatus 10. When the material 27 contacts blood or other bodily fluid within the pseudoaneurysm P, the material 27 (and/or xerogel material within the material 27) may absorb the fluid and become hydrated, causing the material 27, e.g., individual particles, to swell. As previously described, if the material 27 includes a xerogel, the xerogel may hydrate to form a hydrogel and swell to between about two hundred and three thousand percent (200-3000%) of its initial mass within about five to sixty (5-60) seconds. As the material 27 swells, the material 27 may substantially fill the pseudoaneurysm P and at least substantially occlude the mouth of the pseudoaneurysm P communicating with the vessel V, as shown in FIG. 2D. This may relieve pressure, e.g., to prevent further expansion and possible rupture of the pseudoaneurysm P. In addition, as the material 27 may absorb the fluid and/or other materials within the pseudoaneurysm P, e.g., such the blood remains substantially contained by the material 27 within the pseudoaneurysm P, e.g., does not subsequently clot and/or release into the vessel V.

Optionally, before the material 27 is discharged from the apparatus 10, the user may apply pressure to the patient's skin above a region of the injured vessel V upstream from the pseudoaneurysm P. This may temporarily slow or substantially stop flow through the vessel V adjacent the pseudoaneurysm, which may reduce the risk of the material flowing out of the pseudoaneurysm P into the vessel B.

In the embodiment of FIGS. 3A-3C, the sensor 46′ may indicate when the material 27 has been discharged and/or has substantially filled the pseudoaneurysm P, e.g., by detecting laminar or no flow, reduced pressure, and the like.

Turning to FIG. 2D, after the material 27 has been delivered and/or expanded, the apparatus 10 may be retracted proximally from the pseudoaneurysm P and removed from the patient. As described above, in one embodiment, the material 27 may degrade within the body over a period of time, e.g., between about one and ninety (1-90) days or between about five and sixty (5-60) days. In an alternative embodiment, the material 27 may be substantially non-degradable such that the material 27 does not degrade, e.g., within about one to two years. This alternative may desirable when the vessel V is prone to further injury, for example from disease or expected follow-up surgery, so that the vessel V has more time to fully heal.

Optionally, the material 27 may include one or more diagnostic and/or therapeutic agents. For example, the material 27 may include a pro-thrombotic agent, e.g., thrombogin, to enhance clotting of blood within the pseudoaneurysm, a blood thinner to reduce the risk of clotting, antibiotics, agents to enhance healing, and the like. In addition or alternatively, the material 27 may be coated with, may carry, and/or may otherwise include echogenic and/or radiopaque materials, e.g., which may facilitate subsequently monitoring the pseudoaneurysm P, e.g., to confirm that the material 27 has expanded to substantially fill the pseudoaneurysm P.

Although the above methods describe treatment of a pseudoaneurysm, the apparatus and methods described herein for other medical treatments may also be contemplated. For example, the apparatus 10 may be inserted in a bodily tissue region where it is desired to embolize or occlude a vessel, or to reduce blood flow to a region, such as aneurysm sites, arteriovenous malformations, uterine fibroids, and tumors. Additionally, pharmaceutical agents may also be combined with the material 27 to treat infected and/or diseased tissue regions, such as tumors, liver toxins, osteomyelitis, and other conditions in which pharmaceutical treatment is desired and the material 27 may be beneficial to and/or cooperative with such treatment.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. An apparatus for treating a pseudoaneurysm or other body cavity, comprising:
an elongate body comprising a proximal end, a distal end sized for introduction through tissue into a pseudoaneurysm or other body cavity, and a lumen communicating with an outlet on the distal end; and
a plurality of particles within the lumen and dischargeable through the outlet into a pseudoaneurysm or other body cavity, the particles configured for absorbing fluid within the pseudoaneurysm or other body cavity to cause the particles to expand to substantially fill the pseudoaneurysm or other body cavity.

2. The apparatus of claim 1, wherein the particles comprise xerogel.

3. The apparatus of claim 2, wherein the xerogel has a rate of magnitude of expansion between about two and fifty (2-50) times the initial volume when exposed to an aqueous environment.

4. The apparatus of claim 2, wherein the xerogel has a rate of swelling such that, when exposed to an aqueous environment, the xerogel expands between about two hundred and three thousand percent (200-3000%) of the initial mass within about five to sixty (5-60) seconds.

5. The apparatus of claim 2, wherein the xerogel has a density between 0.05 and 0.90 grams per cubic centimeter (g/cc) when disposed within the lumen.

6. The apparatus of claim 2, wherein the xerogel comprises a freeze-dried hydrogel.

7. The apparatus of claim 1, further comprising a plunger slidable within the elongate body for discharging the particles from the lumen.

8. The apparatus of claim 7, wherein the lumen extends between the proximal and distal ends of the elongate body, and wherein the plunger comprises a distal end disposed within the lumen adjacent the particles, and a proximal end extending from the elongate body, the plunger proximal end being depressible for causing the plunger distal end to deliver the particles out the outlet from the lumen.

9. The apparatus of claim 1, further comprising a bleed-back channel extending proximally from the distal end to proximal end of the elongate body, the bleed-back channel configured to cause fluid from within a pseudoaneurysm or other body cavity to pass therethrough to provide a visual indication when the distal end is disposed within the pseudoaneurysm or other body cavity.

10. (canceled)

11. (canceled)

12. The apparatus of claim 1, further comprising a sensor on the distal end for detecting at least one of fluid flow and pressure to provide an indication when the distal end is disposed adjacent to or within a pseudoaneurysm or other body cavity having fluid flow therein.

13. The apparatus of claim 12, wherein the sensor comprises a flow sensor for detecting turbulent flow adjacent the distal end.

14. The apparatus of claim 13, further comprising an output device for providing a visual indication distinguishing between the sensor detecting laminar flow and turbulent flow.

15. The apparatus of claim 12, wherein the sensor comprises a pressure sensor for detecting a pressure differential that indicates when the distal end is disposed within a pseudoaneurysm or other body cavity having an internal pressure greater than ambient pressure.

16. An apparatus for treating a pseudoaneurysm or other body cavity, comprising:
an elongate body comprising a proximal end, a distal end sized for introduction into a pseudoaneurysm or other body cavity, a delivery lumen extending between the proximal and distal ends, and a bleed-back channel extending between the distal end a proximal opening;
a plurality of particles within the delivery lumen and dischargeable through an outlet at the distal end, the particles comprising a xerogel that absorbs fluid within the pseudoaneurysm or other body cavity to cause the particles to hydrate into a hydrogel that expands to substantially fill the pseudoaneurysm or other body cavity; and
an actuator operable from the proximal end for discharging the particles from the outlet into a pseudoaneurysm or other body cavity.

17-21. (canceled)

22. A method for treating a pseudoaneurysm within a patient's body, comprising:
percutaneously inserting a distal end of a delivery device into tissue having the pseudoaneurysm therein;
inserting the distal end of the delivery device into the pseudoaneurysm;
delivering a plurality of particles from the delivery device into the pseudoaneurysm, the particles absorbing fluid within the pseudoaneurysm, thereby causing the particles to expand and substantially fill the pseudoaneurysm; and
removing the delivery device from the patient's body after the particles are delivered into the pseudoaneurysm.

23. The method of claim 22, wherein the xerogel comprise freeze-dried hydrogel.

24. The method of claim 22, wherein the delivery device comprises a bleed-back channel extending proximally from the distal end and wherein inserting the distal end of the delivery device into the pseudoaneurysm comprises monitoring when fluid exits from the bleed-back channel to confirm that the distal end is inserted into the pseudoaneurysm.

25. The method of claim 22, wherein inserting the distal end of the delivery device into the pseudoaneurysm comprises monitoring output from a sensor on the distal end, the output identifying when fluid adjacent the distal end is flowing under turbulent flow conditions, thereby indicating that the distal end is adjacent or within the pseudoaneurysm.

26. The method of claim 22, further comprising applying pressure to the patient's skin upstream to a vessel communicating with the pseudoaneurysm to reduce or substantially cease blood flow adjacent the pseudoaneurysm.

27. A method for treating a pseudoaneurysm communicating with a vessel, comprising:
introducing a distal end of a delivery device carrying xerogel particles into tissue adjacent the pseudoaneurysm;
monitoring introduction of the distal end using an element on the distal end until the element provides an indication that the distal end is within the pseudoaneurysm; and
delivering the particles from the delivery device into the pseudoaneurysm, the particles absorbing fluid within the pseudoaneurysm, thereby causing the particles to hydrate and expand to form a hydrogel that substantially fills the pseudoaneurysm.

28. The method of claim 27, wherein the element comprises an inlet port of a bleed-back channel, and wherein monitoring introduction of the distal end using the element comprises visually monitoring when blood passes from the inlet port proximally through the bleed-back channel.

29. (canceled)

30. The method of claim 27, wherein the element comprises a flow sensor, and wherein monitoring introduction of the distal end using the element comprises detecting a turbulent flow condition adjacent the distal end to indicate that the distal end is disposed adjacent or within the pseudoaneurysm.

31-34. (canceled)

35. A method for treating a pseudoaneurysm within a patient's body, comprising:
percutaneously inserting a distal end of a delivery device into tissue having the pseudoaneurysm therein;
inserting the distal end of the delivery device into the pseudoaneurysm;
delivering xerogel material from the delivery device into the pseudoaneurysm, the xerogel material absorbing fluid within the pseudoaneurysm, thereby hydrating and expanding to form a hydrogel that substantially fills the pseudoaneurysm; and
removing the delivery device from the patient's body after the xerogel material is delivered into the pseudoaneurysm.

36. The method of claim 35, wherein the xerogel material comprises particles.

37. The method of claim 35, wherein the xerogel material comprises a flowable material that is delivered from the delivery device by extruding the flowable material from the delivery device.
